(19) United States
(12) Patent Application Publication
(10) Pub. No.: US 2023/0024422 A1
(43) Pub. Date: Jan. 26, 2023

(54) RESIST TOPCOAT COMPOSITION, AND METHOD OF FORMING PATTERNS USING THE COMPOSITION

(71) Applicants: SAMSUNG SDI CO., LTD., Yongin-si (KR); SAMSUNG ELECTRONICS CO., LTD., Suwon-si (KR)

(72) Inventors: Ran NAMGUNG, Suwon-si (KR); Hyeon PARK, Suwon-si (KR); Minsoo KIM, Suwon-si (KR); Daeseok SONG, Suwon-si (KR); Minki CHON, Suwon-si (KR); Jun Soo KIM, Hwaseong-si (KR); Hyun-Woo KIM, Seongnam-si (KR); Hyun-Ji SONG, Anyang-si (KR); Young Joo CHOI, Hwaseong-si (KR); Suk-Koo HONG, Hwaseong-si (KR)

(21) Appl. No.: 17/746,811

(22) Filed: May 17, 2022

(30) Foreign Application Priority Data
Jul. 1, 2021 (KR) 10-2021-0086530

(51) Int. Cl. C09D 133/08; C09D 133/16; C07C 309/80; C07C 53/18; C07C 53/21; C07C 53/23

(52) U.S. Cl. CPC C09D 133/08; C09D 133/16; C07C 309/80; C07C 53/18; C07C 53/21; C07C 53/23

(57) ABSTRACT

A resist topcoat composition includes an acrylic polymer including a structural unit containing a hydroxy group and a fluorine; a mixture including a sulfonic acid compound containing at least one fluorine and a carboxylic acid compound containing at least one fluorine in a weight ratio of about 1:0.1 to about 1:50; and a solvent. A method of forming patterns uses the resist topcoat composition to form a topcoat over a patterned substrate.

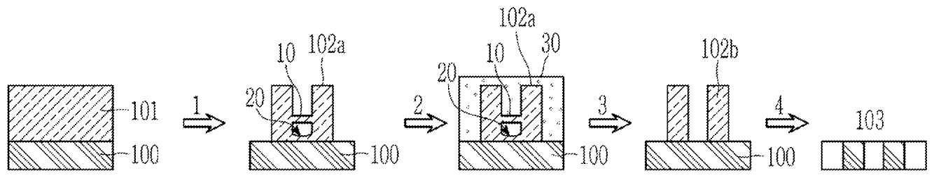

### CROSS-REFERENCE TO RELATED APPLICATION

This application claims priority to and the benefit of Korean Patent Application No. 10-2021-0086530, filed in the Korean Intellectual Property Office on Jul. 1, 2021, the entire content of which is incorporated herein by reference.

### BACKGROUND

### 1. Field

One or more aspects of embodiments of the present disclosure relate to a resist topcoat composition, and a method of forming patterns utilizing the same.

### 2. Description of the Related Art

Recently, the semiconductor industry has developed to the point of using ultrafine techniques providing patterns of several to several tens of nanometers in size (e.g., on nanometer scale). Such ultrafine techniques need effective lithographic techniques.

A lithographic technique in the art involves forming a material layer on a semiconductor substrate, coating a photoresist layer thereon, exposing and developing the photoresist layer to form a photoresist pattern, and then etching the material layer utilizing the photoresist pattern as a mask.

As lithographic techniques are developed, a degree of pattern integration is increasing, and materials and technologies for solving one or more suitable problems occurring in this process are desired. In particular, when a photoresist is patterned using extreme ultraviolet (EUV) as a light source, a high-resolution pattern may be realized, but single line open (SLO) defects may randomly occur on the pattern due to photon shot noise. These SLO defects may lower yield, and thus improved technology solutions are desired.

### SUMMARY

One or more aspects of embodiments of the present disclosure are directed toward a resist topcoat composition capable of not only realizing high-resolution patterns, but also removing single line open (SLO) defects to improve yield.

One or more aspects of embodiments of the present disclosure are directed toward a method of forming patterns utilizing the resist topcoat composition.

One or more embodiments of the present disclosure provide a resist topcoat composition including (a) an acrylic polymer including a structural unit containing a hydroxy group and a fluorine; (b) a mixture including a sulfonic acid compound containing at least one fluorine and a carboxylic acid compound containing at least one fluorine, the sulfonic acid compound and the carboxylic acid compound being in a weight ratio of about 1:0.1 to about 1:50; and (c) a solvent.

The acrylic polymer and the mixture may be included in a weight ratio of about 3:1 to about 30:1.

A total weight of the acrylic polymer and the mixture may be included in an amount of about 0.1 wt % to about 10 wt % based on the total weight of the resist topcoat composition.

The acrylic polymer may include a structural unit represented by Chemical Formula 1:

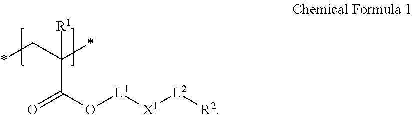

In Chemical Formula 1,

R¹ may be hydrogen or a substituted or unsubstituted C1 to C10 alkyl group,

R² may be hydrogen, a fluorine, a hydroxy group, or a substituted or unsubstituted C1 to C20 alkyl group,

L¹ and L² may each independently be a single bond or a substituted or unsubstituted C1 to C10 alkylene group,

X¹ may be a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group),

R², L¹, and L² includes (e.g., together include) a fluorine and a hydroxy group, and

* is a linking point.

The structural unit of the acrylic polymer is represented by Chemical Formula 2:

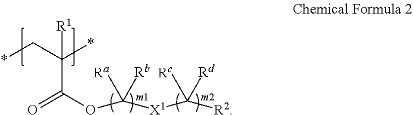

In Chemical Formula 2,

R¹ may be hydrogen or a substituted or unsubstituted C1 to C10 alkyl group,

R^{a}, R^{b}, R^{c}, R^{d}, and R² may each independently be hydrogen, a fluorine, a hydroxy group, or a substituted or unsubstituted C1 to C20 alkyl group,

m1 and m2 may each independently be \an integer from 1 to 10,

X¹ is a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group),

R^{a}, R^{b}, R^{c}, R^{d}, and R² include (e.g., together include) a fluorine and a hydroxy group, and

* is a linking point.

The structural unit containing the hydroxy group and fluorine may be selected from Group I:

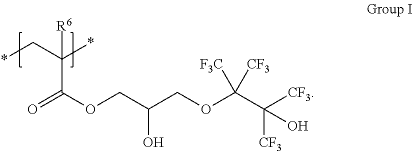

In Group I, R³ to R⁶ may each independently be hydrogen or a methyl group, and * is a linking point.

A weight average molecular weight of the acrylic polymer may be about 1,000 g/mol to about 50,000 g/mol.

The mixture may include two types (classes) of acid compounds, respectively selected from compounds represented by Chemical Formula 3 and Chemical Formula 4.

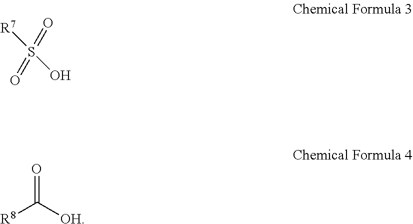

In Chemical Formula 3 and Chemical Formula 4,

R⁷ and R⁸ may each independently be a fluorine, a C1 to C20 alkyl group substituted with at least one fluorine, a C2 to C20 alkenyl group substituted with at least one fluorine, a C2 to C20 alkynyl group substituted with at least one fluorine, a C3 to C20 cycloalkyl group substituted with at least one fluorine, a C3 to C20 cycloalkenyl group substituted with at least one fluorine, a C3 to C20 cycloalkynyl group substituted with at least one fluorine, a C6 to C20 aryl group substituted with at least one fluorine, or a C1 to C20 heteroaryl group substituted with at least one fluorine.

For example, the mixture may include the compound represented by Chemical Formula 3 and the compound represented by Chemical Formula 4.

For example, the compound represented by Chemical Formula 3 may be one selected from compounds of Group II, and the compound represented by Chemical Formula 4 may be one selected from compounds of Group III.

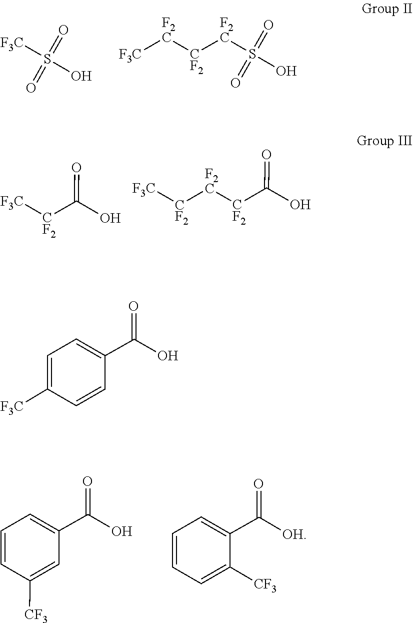

The solvent may be an ether-based solvent represented by Chemical Formula 7.

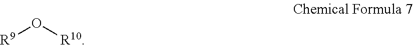

In Chemical Formula 7,

R⁹ and R¹⁰ may each independently be a substituted or unsubstituted C3 to C20 alkyl group.

The ether-based solvent may be selected from diisopropylether, dipropylether, diisoamylether, diamylether, dibutylether, diisobutylether, di-sec-butylether, dihexylether, bis(2-ethylhexyl)ether, didecylether, diundecylether, didodecylether, ditetradecylether, hexadecylether, butylmethylether, butylethylether, butylpropylether, tert-butylmethylether, tert-butylethylether, tert-butylpropylether, di-tert-butylether, cyclopentylmethylether, cyclohexylmethylether, cyclopentylethylether, cyclohexylethylether, cyclopentylpropylether, cyclopentyl-2-propylether, cyclohexylpropylether, cyclohexyl-2-propylether, cyclopentylbutylether, cyclopentyl-tert-butylether, cyclohexylbutylether, cyclohexyl-tert-butylether, 2-octanone, 4-heptanone, and combinations thereof.

One or more embodiments of the present disclosure provide a method of forming patterns, the method including: forming a photoresist pattern on a substrate, coating the aforementioned resist topcoat composition on the photoresist pattern, drying and heating the substrate on which the resist topcoat composition is coated to form a topcoat, and spraying a rinse solution on the substrate coated with the topcoat to remove the topcoat.

The heating of the substrate coated with the resist topcoat composition may be performed at a temperature of about 100° C. to about 500° C.

The resist topcoat composition according to an embodiment may have excellent or suitable solubility in a solvent having low reactivity with respect to photoresist, and thus may effectively remove SLO defects without loss of photoresist fine patterns.

The simple process for removing SLO defects may be advantageous in terms of process economy. Accordingly, the resist topcoat composition according to an embodiment or a pattern prepared therefrom may be advantageously utilized to form a fine pattern of a photoresist utilizing a high energy light source (such as EUV).

### BRIEF DESCRIPTION OF THE DRAWING

The drawing is a cross-sectional view for explaining a method of forming patterns utilizing a resist topcoat composition according to an embodiment.

### DETAILED DESCRIPTION

Example embodiments of the present disclosure will hereinafter be described in more detail, and may be easily performed by a person skilled in the art. However, this disclosure may be embodied in many different forms and should not be construed as limited to the example embodiments set forth herein.

In the drawings, the thickness of layers, films, panels, regions, etc., may be exaggerated for clarity, and like reference numerals designate like elements throughout the specification. It will be understood that when an element such as a layer, film, region, or substrate is referred to as being “on” another element, it can be directly on the other element, or intervening elements may also be present. In contrast, when an element is referred to as being “directly on” another element, there are no intervening elements present.

As used herein, when a definition is not otherwise provided, “substituted” refers to replacement of a hydrogen atom (e.g., in a compound, group, or moiety) by a non-hydrogen atom substituent selected from a halogen atom (F, Br, Cl, and/or I), a hydroxy group, an alkoxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid group or a salt thereof, a vinyl group, a C1 to C20 alkyl group, a C2 to C20 alkenyl group, a C2 to C20 alkynyl group, a C6 to C30 aryl group, a C7 to C30 arylalkyl group, a C6 to C30 allyl group, a C1 to C30 alkoxy group, a C1 to C20 heteroalkyl group, a C3 to C20 heteroarylalkyl group, a C3 to C30 cycloalkyl group, a C3 to C15 cycloalkenyl group, a C6 to C15 cycloalkynyl group, a C3 to C30 heterocycloalkyl group, and combinations thereof.

As used herein, when a definition is not otherwise provided, “hetero” refers to the inclusion of 1 to 10 heteroatoms selected from nitrogen (N), oxygen (O), sulfur (S), and phosphorus (P).

In the present specification, the term “acrylic polymer” refers to an acrylic polymer and a methacrylic polymer.

Unless otherwise specified in the present specification, the weight average molecular weight may be measured by dissolving a powder sample in tetrahydrofuran (THF) and then utilizing an Agilent 1200 series Gel Permeation Chromatography (GPC) (column is Shodex Company LF-804, standard sample is Shodex company polystyrene).

Unless otherwise defined in the specification, “*” indicates a linking point of a structural unit or a compound moiety of a compound.

As used herein, singular forms such as “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively. As used herein, expressions such as “at least one of,” “one of,” and “selected from,” when preceding a list of elements, modify the entire list of elements and do not modify the individual elements of the list. As used herein, the term “and/or” includes any and all combinations of one or more of the associated listed items.

The term “may” will be understood to refer to “one or more embodiments,” some of which include the described element and some of which exclude that element and/or include an alternate element. Similarly, alternative language such as “or” refers to “one or more embodiments,” each including a corresponding listed item.

Hereinafter, a resist topcoat composition according to an embodiment is described.

One or more embodiments of the present disclosure relate to a resist topcoat composition capable of improving photoresist patterning (e.g., having improved photoresist patterning) by adding a simple process during the fine pattern forming process of photolithography utilizing a short-wavelength light source (such as an ArF excimer laser (wavelength: 193 nm)) or high energy rays (such as extreme ultraviolet (EUV; wavelength: 13.5 nm)) to remove SLO defects in the resist pattern, and a method for forming a photoresist pattern utilizing such a topcoat.

The resist topcoat composition according to an embodiment includes an acrylic polymer including a structural unit containing a hydroxy group and a fluorine; a mixture including a sulfonic acid compound containing at least one fluorine and a carboxylic acid compound containing at least one fluorine in a weight ratio of about 1:0.1 to about 1:50; and a solvent.

The composition according to embodiments is coated on the photoresist, and has excellent or suitable solubility in a solvent having low reactivity to (e.g., solubility for) the photoresist, so that it may be easily removed together with the SLO defects, which is advantageous for realizing high resolution.

The formation and removal of the resist topcoat are simple processes, which is advantageous in terms of process economy, and the yield may be improved by the removal of the SLO defects.

Because the acrylic polymer included in the composition contains a hydroxy group and fluorine in the structural unit at the same time (e.g., simultaneously), it has excellent or suitable solubility in a solvent, may be uniformly coated on a pattern, and may have minimal or reduced influence (impact) on the photoresist.

In some embodiments, the mixture included in the composition may include two types (classes) of acid compounds, for example, a sulfonic acid compound containing at least one fluorine, and a carboxylic acid compound containing at least one fluorine, in a weight ratio of about 1:0.1 to about 1:50.

As described above, when a mixture containing two types (classes) of acid compounds is added, defect portions of the resist may be selectively removed.

For example, the mixture including the two acid compounds may include a sulfonic acid compound containing at least one fluorine and a carboxylic acid compound containing at least one fluorine.

The mixture may include the two types (classes) of acid compounds (e.g., the sulfonic acid compound and the carboxylic acid compound) in a weight ratio of about 1:0.3 to about 1:40, about 1:0.3 to about 1:35, or about 1:1 to about 1:30.

When the mixing ratio is within the above range, the SLO defect removal effect may be further improved.

Therefore, by utilizing the resist topcoat composition according to an embodiment, a high-resolution pattern may be obtained with a high yield.

In some embodiments, the acrylic polymer and the mixture may be included in a weight ratio of about 3:1 to about 30:1, for example, about 3:1 to about 25:1, or about 3:1 to about 20:1.

By including the acrylic polymer and the mixture in the above weight ratio, the resist topcoat composition according to an embodiment may provide an resist topcoat that is easy for SLO defect removal.

A total weight of the acrylic polymer and the mixture may be about 0.1 wt % to about 10 wt % based on the total weight of the resist topcoat composition. Within the above range, resist topcoat removal may be easy.

For example, the acrylic polymer may include a structural unit represented by Chemical Formula 1:

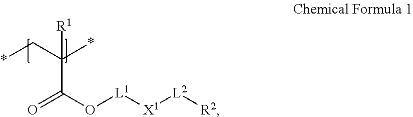

In Chemical Formula 1,

R¹ may be hydrogen or a substituted or unsubstituted C1 to C10 alkyl group,

R² may be hydrogen, a fluorine, a hydroxy group, or a substituted or unsubstituted C1 to C20 alkyl group,

L¹ and L² may each independently be a single bond or a substituted or unsubstituted C1 to C10 alkylene group,

X¹ may be a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group), and

R², L¹, and L² together include a fluorine and a hydroxy group.

The statement that “R², L¹, and L² together include a fluorine and the hydroxy group” may indicates that at least one fluorine and at least one hydroxy group are included within the combination of R², L¹, and L², for example:

R² may be a C1 to C10 alkyl group substituted with at least one fluorine and at least one hydroxy group, or

at least one of L¹ or L² may be a C1 to C10 alkylene group substituted with at least one fluorine and at least one hydroxy group, or

one of L¹ or L² may be a C1 to C10 alkylene group substituted with at least one fluorine and the other may be a C1 to C10 allylene group substituted with at least one hydroxy group, or

R² may be a fluorine and at least one of L¹ or L² may each independently be a C1 to C10 alkylene group substituted with a hydroxy group, or

R² may be a hydroxy group and at least one of L¹ or L² may each independently be a C1 to C10 alkylene group substituted with a fluorine, or

R² may be a C1 to C20 alkyl group substituted with at least one hydroxy group and at least one fluorine.

For example, the acrylic polymer may include a structural unit represented by Chemical Formula 2:

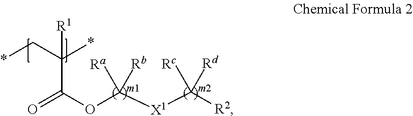

In Chemical Formula 2,

R¹ may be hydrogen or a substituted or unsubstituted C1 to C10 alkyl group,

R^{a}, R^{b}, R^{c}, R^{d}, and R² may each independently be hydrogen, a fluorine, a hydroxy group, or a substituted or unsubstituted C1 to C20 alkyl group,

m1 and m2 may each independently be an integer from 1 to 10,

X¹ may be a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group), and

R^{a}, R^{b}, R^{c}, R^{d}, and R² together include a fluorine and a hydroxy group.

The statement that “R^{a}, R^{b}, R^{c}, R^{d}, and R² together include a fluorine and a hydroxy group” indicates that at least one fluorine and at least one hydroxy group are included within the combination of R^{a}, R^{b}, R^{C}, R^{d}, and R², for example:

at least one of R^{a}, R^{b}, R^{c}, R^{d}, or R² may be a C1 to C10 alkyl group substituted with a fluorine and at least one of the remaining groups may each independently be a C1 to C10 alkyl group substituted with a hydroxy group, or

at least one of R^{a}, R^{b}, R^{c}, R^{d}, or R² may each independently be a C1 to C10 alkyl group substituted with a hydroxy group and a fluorine, or

at least one of R^{a}, R^{b}, R^{c}, R^{d}, or R² may each independently be a C1 to C20 alkyl group substituted with a hydroxy group and a fluorine, or

at least one of R^{a}, R^{b}, R^{c}, or R² may be a fluorine and at least one of the remaining groups may be a hydroxy group, or

at least one of R^{a}, R^{b}, R^{c}, R^{d}, or R² may be a fluorine and at least one of the remaining groups may be a C1 to C10 alkyl group substituted with a hydroxy group, or

at least one of R^{a}, R^{b}, R^{c}, R^{d}, or R² may be a hydroxy group and at least one of the remaining groups may be a C1 to C10 alkyl group substituted with a fluorine, or

at least one of R^{a}, R^{b}, R^{c}, R^{d}, or R² may be a C1 to C20 alkyl group substituted with a fluorine and at least one of the remaining groups may be a C1 to C20 alkyl group substituted with a hydroxy group.

For example, R¹ may be hydrogen or a methyl group,

X¹ may be a single bond or —O—, and

R² may be a fluorine, a hydroxy group, a C1 to C10 alkyl group substituted with at least one fluorine, or a C1 to C10 alkyl group substituted with at least one hydroxy group.

For example, R^{c}, R^{d}, and R² of Chemical Formula 2 may together include a fluorine and a hydroxy group.

For example, at least one of R^{c} or R^{d} in Chemical Formula 2 may be a fluorine or a C1 to C10 alkyl group substituted with at least one fluorine, and R² may be a hydroxy group or a C1 to C10 alkyl group substituted with at least one hydroxy group.

For example, at least one of R^{c} or R^{d} in Chemical Formula 2 may be a hydroxy group or a C1 to C10 alkyl group substituted with at least one hydroxy group, and R² may be a fluorine or a C1 to C10 alkyl group substituted with at least one fluorine.

For example, in Chemical Formula 2, R^{c} may be a hydroxy group or a C1 to C10 alkyl group substituted with at least one hydroxy group, R^{d} may be a fluorine or a C1 to C10 alkyl group substituted with at least one fluorine, and R² may be a hydroxy group, a fluorine or a C1 to C10 alkyl group substituted with at least one fluorine or at least one hydroxy group.

For example, at least one of R^{c} or R^{d} of Chemical Formula 2 may be a fluorine or a C1 to C10 alkyl group substituted with at least one fluorine, R² may be a hydroxy group, or a C1 to C10 alkyl group substituted with at least one hydroxy group and at least one fluorine.

For example, the structural unit containing the hydroxy group and fluorine may be selected from Group I.

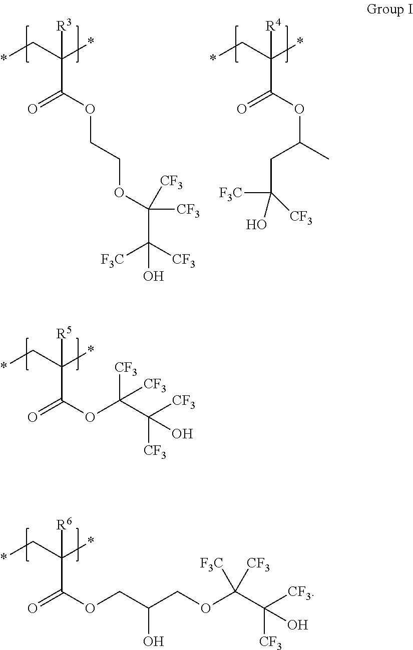

In Group I, R³ to R⁶ may each independently be hydrogen or a methyl group, and * is a linking point.

The acrylic polymer may have a weight average molecular weight (Mw) of about 1,000 g/mol to about 50,000 g/mol. For example, it may have a weight average molecular weight of about 2,000 g/mol to about 30,000 g/mol, for example, about 3,000 g/mol to about 20,000 g/mol, or for example, about 4,000 g/mol to about 10,000 g/mol, but not limited thereto. When the weight average molecular weight of the acrylic polymer is within the above ranges, a carbon content (e.g., amount) and solubility in a solvent of the resist topcoat composition including the polymer may be enhanced and/or optimized.

For example, the mixture may include two types (classes) of acid compounds selected from a compound represented by Chemical Formula 3 and a compound represented by Chemical Formula 4:

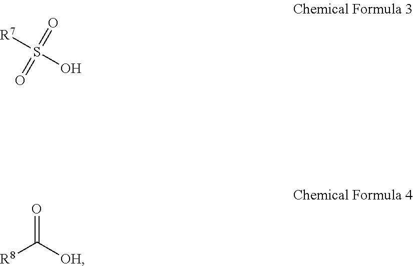

In Chemical Formula 3 and Chemical Formula 4,

R⁷ and R⁸ may each independently be a fluorine, a C1 to C20 alkyl group substituted with at least one fluorine, a C2 to C20 alkenyl group substituted with at least one fluorine, a C2 to C20 alkynyl group substituted with at least one fluorine, a C3 to C20 cycloalkyl group substituted with at least one fluorine, a C3 to C20 cycloalkenyl group substituted with at least one fluorine, a C3 to C20 cycloalkynyl group substituted with at least one fluorine, a C6 to C20 aryl group substituted with at least one fluorine, or a C1 to C20 heteroaryl group substituted with at least one fluorine.

For example, R⁷ and R⁸ of Chemical Formula 3 and Chemical Formula 4 may each independently be a C1 to C10 alkyl group substituted with at least one fluorine or a C6 to C20 aryl group substituted with at least one fluorine.

In an example embodiment, the mixture may include the compound represented by Chemical Formula 3 and the compound represented by Chemical Formula 4.

For example, the compound represented by Chemical Formula 3 may be one selected from the compounds of Group II, and the compound represented by Chemical Formula 4 may be one selected from the compounds of Group III:

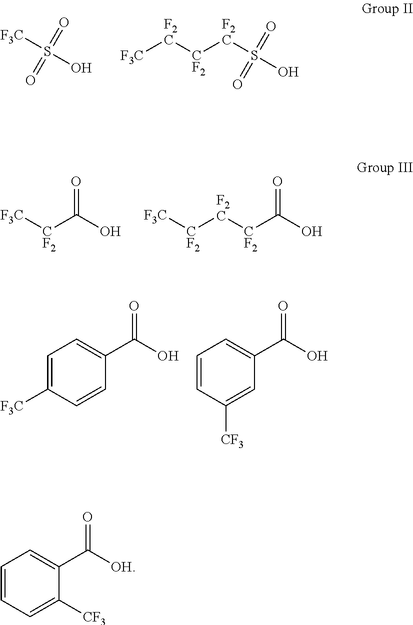

In some embodiments, the resist topcoat composition may further include at least one other polymer selected from an epoxy-based resin, a novolac-based resin, a glycoluril-based resin, and a melamine-based resin, but the present disclosure is not limited thereto.

The resist topcoat composition may further include an additive including a surfactant, a thermal acid generator, a plasticizer, or a combination thereof.

The surfactant may be, for example, an alkylbenzene sulfonic acid salt, an alkylpyridinium salt, polyethylene glycol, a quaternary ammonium salt, and/or the like, but is not limited thereto.

The thermal acid generator may be, for example, an acid compound (such as p-toluene sulfonic acid, trifluoromethanesulfonic acid, pyridinium p-toluene sulfonic acid, salicylic acid, sulfosalicylic acid, citric acid, benzoic acid, hydroxybenzoic acid, naphthalene carboxylic acid and/or benzointosylate, 2-nitrobenzyl tosylate, and other organic sulfonic acid alkyl esters), but is not limited thereto.

The additive may be included in an amount of about 0.001 to about 40 parts by weight based on 100 parts by weight of the resist topcoat composition. Within the above range, solubility may be improved without changing the optical properties of the resist topcoat composition.

The solvent may be an ether-based solvent represented by Chemical Formula 7:

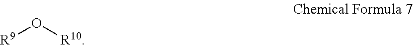

In Chemical Formula 7,

R⁹ and R¹⁰ may each independently be a substituted or unsubstituted C3 to C20 alkyl group.

For example, the ether-based solvent may be selected from diisopropyl ether, dipropyl ether, diisoamyl ether, diamyl ether, dibutyl ether, diisobutyl ether, di-sec-butyl ether, dihexyl ether, bis(2-ethylhexyl) ether, didecyl ether, diundecyl ether, didodecyl ether, ditetradecyl ether, hexadecyl ether, butyl methyl ether, butyl ethyl ether, butyl propyl ether, tert-butyl methyl ether, tert-butyl ethyl ether, tert-butylpropyl ether, di-tert-butyl ether, cyclopentylmethyl ether, cyclohexylmethyl ether, cyclopentylethyl ether, cyclohexylethyl ether, cyclopentylpropyl ether, cyclopentyl-2-propyl ether, cyclohexylpropyl ether, cyclohexyl-2-propyl ether, cyclopentylbutyl ether, cyclopentyl-tert-butyl ether, cyclohexylbutyl ether, cyclohexyl-tert-butyl ether, 2-octanone, 4-heptanone, and combinations thereof.

The ether-based solvent may have sufficient solubility or dispersibility for the aforementioned composition.

According to another embodiment, a photoresist pattern may be prepared utilizing the aforementioned resist topcoat composition. The resist topcoat may be in a cured film through a heat treatment process after coating the aforementioned resist topcoat composition on, for example, a photoresist pattern.

Hereinafter, a method of forming patterns utilizing the aforementioned resist topcoat composition is described with reference to the drawing.

A method of forming patterns according to an embodiment includes forming a photoresist pattern (e.g., a preliminary photoresist pattern) 102a on a substrate 100 (act 1), coating the aforementioned resist topcoat composition on the preliminary photoresist pattern 102a, drying and heating the substrate 100 on which the resist topcoat composition is coated to form a topcoat 30 (act 2), and spraying a rinse solution on the substrate coated with the topcoat to remove the topcoat 30 (act 3).

The forming of the preliminary photoresist pattern 102a on the substrate 100 (act 1) may include coating a semiconductor resist composition on the substrate 100 by spin coating, slit coating, inkjet printing, etc., and drying and heat treating the coated semiconductor photoresist composition to form a photoresist film 101, and selectively exposing and developing the photoresist film 101 to dissolve and remove the photoresist film corresponding to the exposed area to form a preliminary photoresist pattern 102a.

The forming of the preliminary photoresist pattern 102a may be performed by any suitable method, and details thereof will not be provided.

In the preliminary photoresist pattern 102a formed in this way, defects such as the bridge 10 connecting adjacent pattern(s) and the scum 20 remaining in the gap between the patterns may occur, which may cause later formation of SLO defects in the thin film pattern, and thereby cause a decrease of yield.

In the method of forming patterns according to an embodiment, in order to remove the bridge 10 and the scum 20 after the photoresist pattern is formed, the method may further include coating the aforementioned resist topcoat composition over the preliminary photoresist pattern 102a; drying and heating the substrate 100 coated with the resist topcoat composition to form a topcoat 30 (act 2); and spraying a rinse solution on the substrate 100 coated with the topcoat 30 to remove the topcoat 30 (act 3).

The heating of the substrate 100 coated with the resist topcoat composition may be performed at a temperature of about 100° C. to about 500° C.

In the removing of the topcoat 30 by spraying a rinse solution, a solvent having low reactivity with respect to the photoresist and high solubility with respect to the topcoat may be advantageously utilized.

As such, in the photoresist pattern 102b formed after performing the coating of the topcoat 30 (act 2) and removing of the topcoat 30 (act 3), the bridge 10 and the scum 20 may be removed, compared with the photoresist pattern (e.g., a preliminary photoresist pattern) 102a before performing the processes (2) and (3), so that the patterning of the photoresist may be improved.

The thin film pattern 103 may be finally formed through a process (act 4) of etching the exposed thin film of the substrate 100 by applying the photoresist pattern 102b as an etching mask, and in the thin film pattern 103 formed in this way, SLO defects may be effectively removed without loss of the fine pattern.

The thin film may be etched, for example, by dry etching utilizing an etching gas, and the etching gas may be, for example, CHF₃, CF₄, Cl₂, BCl₃, or mixture(s) thereof.

In the exposure process described above, the thin film pattern formed using the photoresist pattern 102b that is formed by exposure process performed using the EUV light source may have a width corresponding to that of the photoresist pattern 102b. For example, the photoresist pattern 102b may have a width of about 5 nm to about 100 nm. For example, the thin film pattern 103 formed from the photoresist pattern 102b that is formed by the exposure process performed utilizing an EUV light source may have a width of about 5 nm to about 90 nm, about 5 nm to about 80 nm, about 5 nm to about 70 nm, about 5 nm to about 60 nm, about 5 nm to about 50 nm, about 5 nm to about 40 nm, about 5 nm to about 30 nm, or about 5 nm to about 20 nm, similar to the photoresist pattern 102b, and in some embodiments, may for example be formed in a width of less than or equal to about 20 nm.

Hereinafter, the present disclosure will be described in more detail through examples relating to the synthesis of the aforementioned polymer and the preparation of a resist topcoat composition including the same. However, the present disclosure is not limited to or by the following examples.

### Synthesis Examples

### Synthesis of Acrylic Polymer

### Synthesis Example 1: Synthesis of Monomer

20 g (59.86 mmol) of hexafluoro-2,3-bis(trifluoromethyl)-2,3-butanediol(perfluoropinacol), 7.79 g (59.86 mmol) of 2-(hydroxyethyl)methacrylate, and 18.84 g (71.84 mmol) of triphenylphosphine (Ph₃P) were mixed in 110 mL of diethyl ether under a nitrogen atmosphere and then stirred. After stirring for 30 minutes, the mixture was cooled down to 0° C., and another mixture of 14.52 g (71.84 mmol) of diisopropylazodicarboxylate (DIAD) and 35 mL of diethyl ether was slowly added thereto over 2 hours. Subsequently, the obtained mixture was stirred at room temperature for 24 hours, and then concentrated. The concentrated mixture was dissolved in dichloromethane, and then treated through column chromatography by utilizing silica gel to separate a synthesized material. The separated material was distilled under a reduced pressure, obtaining 2-[3,3,3-trifluoro-2-hydroxy-1,1,2-tris(trifluoromethyl)propoxy]ethyl 2-methyl-2-propenoate represented by Chemical Formula 1a.

* ¹H-NMR (Acetone-d6): δ1.90 (3H, t), 4.36 (4H, m), 5.63 (1H, t), 6.09 (1H, t), 8.34 (1H, s)

* ¹⁹F-NMR (Acetone-d6): δ−70.12 (6F, m), −65.38 (6F, m)

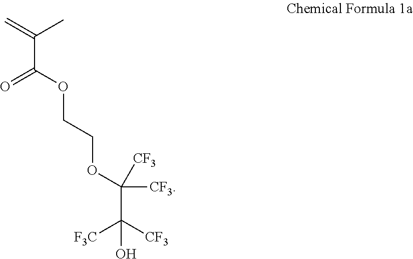 [SMILES: C=C(C)C(=O)OCCOC(C(F)(F)F)(C(F)(F)F)C(O)(C(F)(F)F)C(F)(F)F]

### Synthesis Example 2: Preparation of Polymer P1

The compound represented by Chemical Formula 1a (37.5 g, 84.0 mmol) according to Synthesis Example 1, dimethyl 2,2′-azobis(2-methylpropionate) (2.5 g, 10.9 mmol, Wako Chemical, Inc.), and diisoamyl ether (DIAE, 60 g) (e.g., as monomers) were put in a 500 mL 2-necked round flask under a nitrogen atmosphere, and a condenser was connected thereto. After increasing the temperature to 110° C., the obtained mixture was reacted for 24 hours, and the reaction solution was cooled down to room temperature. The reaction solution was added dropwise with stirring into a 1 L wide-mouth bottle containing 225 g of heptane, producing a gum, and then, a supernatant was removed therefrom. After dissolving the remaining gum in 40 g of DIAE, 180 g of heptane was added thereto to form precipitates, and a supernatant was removed therefrom, which was repeated three times to remove monomers and oligomers.

Finally, 22.5 g of a polymer P1 including a structural unit represented by Chemical Formula 1b (a weight average molecular weight: 4,500) was obtained.

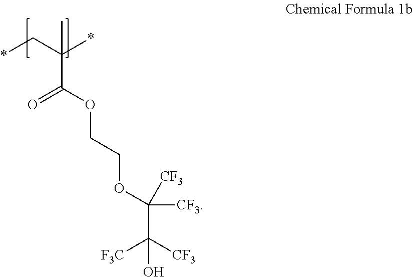

In Chemical Formula 1 b, * is a linking point (e.g., to other units of the copolymer backbone).

### Preparation of Resist Topcoat Composition

### Examples 1 to 12 and Comparative Examples 1 and 2

Each resist topcoat composition according to Examples 1 to 12 and Comparative Examples 1 and 2 was prepared by mixing the acrylic polymer P1 according to Synthesis Example 2, a mixture including two selected from the acid compounds represented by A1 to A6, and diisoamylether (DIAE) in compositions shown in Table 1, stirring the mixture at room temperature (e.g., 23° C.) for 24 hours, and filtering it with a TEFLON (tetrafluoroethylene) filter having a pore size of 0.45 μm.

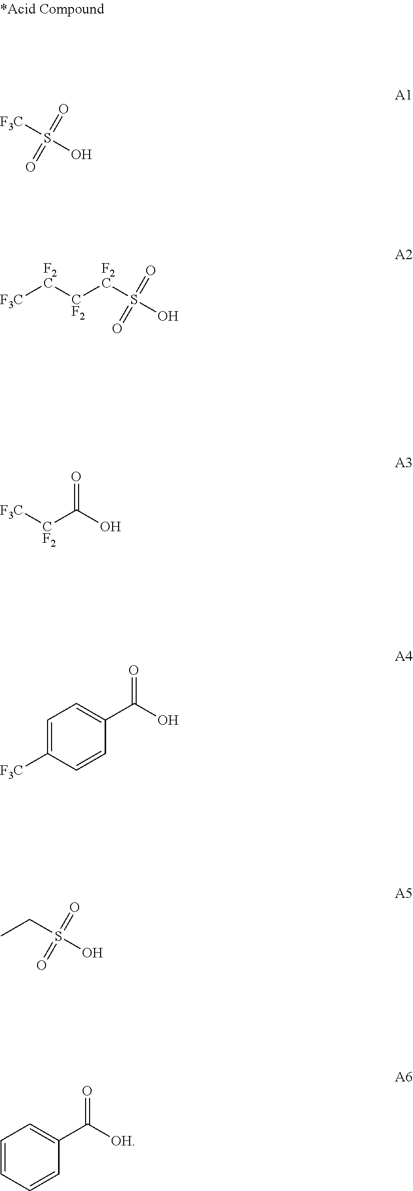

TABLE 1

| | Mixture | | | | | | |
|---|---|---|---|---|---|---|---|
| | First acid | | Second acid | | Acrylic | | |
| | compound | | compound | | polymer | | Solvent |
| | | Content | | Content | | Content | (DIAE) |
| Composition | Type | (wt %) | Type | (wt %) | Type | (wt %) | (wt %) |
| Example 1 | A1 | 0.09 | A2 | 0.65 | P1 | 4.3 | 94.97 |
| Example 2 | A1 | 0.09 | A2 | 1.29 | P1 | 4.3 | 94.32 |
| Example 3 | A1 | 0.13 | A2 | 0.65 | P1 | 4.3 | 94.93 |
| Example 4 | A1 | 0.13 | A2 | 1.29 | P1 | 4.3 | 94.28 |
| Example 5 | A1 | 0.09 | A3 | 0.65 | P1 | 4.3 | 94.97 |
| Example 6 | A1 | 0.09 | A3 | 1.29 | P1 | 4.3 | 94.32 |
| Example 7 | A1 | 0.13 | A3 | 0.65 | P1 | 4.3 | 94.93 |
| Example 8 | A1 | 0.13 | A3 | 1.29 | P1 | 4.3 | 94.28 |
| Example 9 | A1 | 0.09 | A4 | 0.65 | P1 | 4.3 | 94.97 |
| Example 10 | A1 | 0.09 | A4 | 1.29 | P1 | 4.3 | 94.32 |
| Example 11 | A1 | 0.13 | A4 | 0.65 | P1 | 4.3 | 94.93 |
| Example 12 | A1 | 0.13 | A4 | 1.29 | P1 | 4.3 | 94.28 |
| Comparative | A2 | 1.30 | — | — | P1 | 4.3 | 94.40 |
| Example 1 | | | | | | | |
| Comparative | A5 | 0.09 | A6 | 0.22 | P1 | 4.3 | 95.40 |
| Example 2 | | | | | | | |

### Evaluation 1: Evaluation of Solubility

Each photoresist topcoat composition according to Examples 1 to 12 and Comparative Examples 1 and 2 was prepared and stirred for 24 hours, and then observed with naked eyes to check whether or not precipitates were produced, and the results are shown in Table 2.

(No precipitation—Solubility O, with precipitation—Solubility X)

### Evaluation 2: Evaluation of Non-Pattern Wafer (NPW) Strip

Each photoresist topcoat composition was spin-on coated on a silicon substrate coated with a photoresist, and then heat-treated on a hot plate at 110° C. for 1 minute, forming an about 50 nm-thick topcoat for a photoresist. Subsequently, the substrate coated with the topcoat was rinsed with a rinse solution (diisoamylether (DIAE)), heat-treated on a hot plate at 110° C. for 1 minute, and then measured with respect to a thickness change of the photoresist, which was utilized to calculate an NPW strip according to the following equation, and the results are shown in Table 2.

(NPW strip=PR thickness (nm) after forming and rinsing a photoresist topcoat−initial PR thickness (nm))

### Evaluation 3: Evaluation of SLO Defects

On a 12 inch silicon substrate, a substrate composed of (a lower SiON film—a spin-on carbon film—a topcoat) were sequentially formed. On the SiON film, a 1:1 line/space photoresist pattern with a pitch of 36 nm was formed in an EUV lithography method. The photoresist pattern was transferred into the lower SiON film through dry etching utilizing plasma. Then, all defects including bridge defects between the line patterns were inspected with a defect analysis equipment utilizing a deep UV (DUV) laser. The detected defects were classified by utilizing SEM, providing the number of the detected defects per unit area (ea/cm²).

Herein, when the number of SLO defects without utilizing the photoresist topcoat compositions was converted into 100, ‘⊚’ was given to a case that the number of defects was less than or equal to 60%, ‘∘’ was given to a case that the number of defects was greater than 60% and less than or equal to 80%, and ‘X’ was given to a case that the number of defects was greater than 80%.

TABLE 2

| | Solubility | NPW strip | SLO Defects |
|---|---|---|---|
| Example 1 | ◯ | −3.1 | ⊚ |
| Example 2 | ◯ | −2.5 | ⊚ |
| Example 3 | ◯ | −3.6 | ⊚ |
| Example 4 | ◯ | −3.5 | ⊚ |
| Example 5 | ◯ | −3.4 | ⊚ |
| Example 6 | ◯ | −3.4 | ⊚ |
| Example 7 | ◯ | −3.2 | ⊚ |
| Example 8 | ◯ | −3.1 | ⊚ |
| Example 9 | ◯ | −3.3 | ⊚ |
| Example 10 | ◯ | −2.5 | ⊚ |
| Example 11 | ◯ | −3.5 | ⊚ |
| Example 12 | ◯ | −3.3 | ⊚ |
| Comparative Example 1 | ◯ | −2.5 | ◯ |
| Comparative Example 2 | ◯ | −1.3 | X |

Referring to Table 2, when the resist topcoat compositions of Examples 1 to 12 were applied, compared with when the resist topcoat compositions of Comparative Examples 1 and 2 were applied, excellent or suitable solubility, NPW strip (effective when −5.0 nm to −2.5 nm), and defect improvement effects were exhibited.

Embodiments of the present disclosure have been described and illustrated, however, the present disclosure is not limited to embodiments as described, and may be variously modified and transformed without departing from the spirit and scope of the present disclosure, as set forth in the following claims and equivalents thereof.

Description of Some of the Symbols

| | |
|---|---|
| 1: forming a photoresist pattern on a substrate | |
| 2: coating the aforementioned resist topcoat | |
| composition on the photoresist pattern | |
| and drying and heating the substrate on which | |
| the resist topcoat composition is coated | |
| to form a topcoat | |
| 3: spraying a rinse solution on the substrate | |
| coated with the topcoat to remove the | |
| topcoat | |
| 4: etching the exposed thin film by applying | |
| the photoresist pattern as an etching mask | |
| 10: bridge | 20: scum |
| 30: topcoat | |
| 100: substrate | 101: photoresist film |
| 102a: preliminary photoresist pattern formed | |
| before performing coating and removing | |
| the topcoat | |
| 102b: photoresist pattern formed after performing | |
| coating and removing the topcoat | |
| 103: thin film pattern | |

What is claimed is:

1. A resist topcoat composition, the composition comprising:
an acrylic polymer comprising a structural unit comprising a hydroxy group and a fluorine;
a mixture comprising:
a sulfonic acid compound comprising at least one fluorine; and
a carboxylic acid compound comprising at least one fluorine, the sulfonic acid compound and the carboxylic acid compound being in a weight ratio of about 1:0.1 to about 1:50; and
a solvent.

2. The resist topcoat composition of claim 1, wherein the acrylic polymer and the mixture are included in a weight ratio of about 3:1 to about 30:1.

3. The resist topcoat composition of claim 1, wherein a total weight of the acrylic polymer and the mixture is about 0.1 wt % to about 10 wt % based on the total weight of the resist topcoat composition.

4. The resist topcoat composition of claim 1, wherein the acrylic polymer comprises a structural unit represented by Chemical Formula 1: 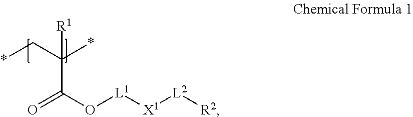
and
wherein, in Chemical Formula 1,
R¹ is hydrogen or a substituted or unsubstituted C1 to C10 alkyl group,
R² is hydrogen, a fluorine, a hydroxy group, or a substituted or unsubstituted C1 to C20 alkyl group,
L¹ and L² are each independently a single bond or a substituted or unsubstituted C1 to C10 alkylene group,
X¹ is a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group),
R², L¹, and L² together comprise a fluorine and a hydroxy group, and
is a linking point.

5. The resist topcoat composition of claim 1, wherein the structural unit of the acrylic polymer is represented by Chemical Formula 2: 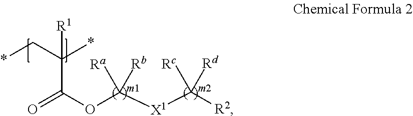
and
wherein, in Chemical Formula 2,
R¹ is hydrogen or a substituted or unsubstituted C1 to C10 alkyl group,
R^{a}, R^{b}, R^{e}, R^{d}, and R² are each independently hydrogen, a fluorine, a hydroxy group, or a substituted or unsubstituted C1 to C20 alkyl group,
m1 and m2 are each independently integer from 1 to 10,
X¹ is a single bond, —O—, —S—, —S(O)—, —S(O)₂—, —C(O)—, —(CO)O—, —O(CO), —O(CO)O—, or —NR′— (wherein, R′ is hydrogen, deuterium, or a C1 to C10 alkyl group),
R^{a}, R^{b}, R^{c}, R^{d}, and R² together comprise a fluorine and a hydroxy group, and
is a linking point.

6. The resist topcoat composition of claim 1, wherein the structural unit comprising the hydroxy group and fluorine is selected from Group I: 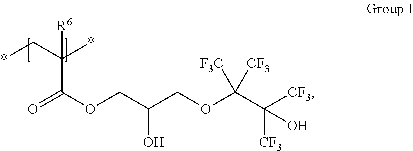
and
wherein, in Group I,
R³ to R⁶ are each independently hydrogen or a methyl group, and
is a linking point.

7. The resist topcoat composition of claim 1, wherein a weight average molecular weight of the acrylic polymer is about 1,000 g/mol to about 50,000 g/mol.

8. The resist topcoat composition of claim 1, wherein the mixture comprises a compound represented by Chemical Formula 3 and a compound represented by Chemical Formula 4: 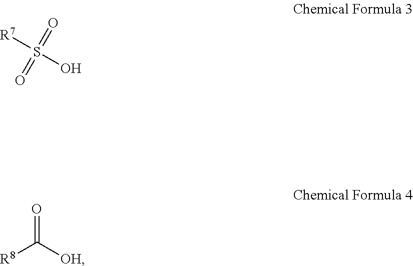
and
wherein in Chemical Formula 3 and Chemical Formula 4,
R⁷ and R⁸ are each independently a fluorine, a C1 to C20 alkyl group substituted with at least one fluorine, a C2 to C20 alkenyl group substituted with at least one fluorine, a C2 to C20 alkynyl group substituted with at least one fluorine, a C3 to C20 cycloalkyl group substituted with at least one fluorine, a C3 to C20 cycloalkenyl group substituted with at least one fluorine, a C3 to C20 cycloalkynyl group substituted with at least one fluorine, at least one a C6 to C20 aryl group substituted with at least one fluorine, or a C1 to C20 heteroaryl group substituted with at least one fluorine\.

9. The resist topcoat composition of claim 8, wherein the mixture comprises the compound represented by Chemical Formula 3 and the compound represented by Chemical Formula 4.

10. The resist topcoat composition of claim 8, wherein the compound represented by Chemical Formula 3 is selected from compounds of Group II and the compound represented by Chemical Formula 4 is selected from compounds of Group III:

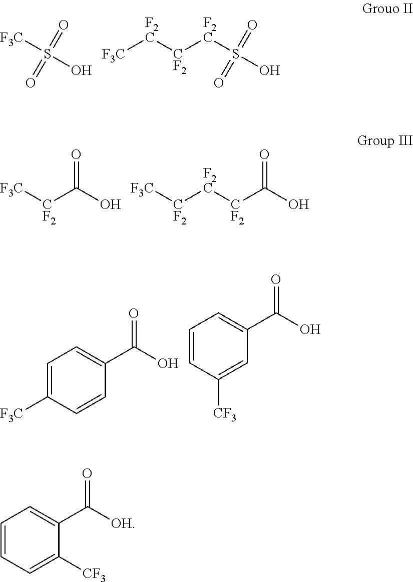

11. The resist topcoat composition of claim 1, wherein the solvent is an ether-based solvent represented by Chemical Formula 7: 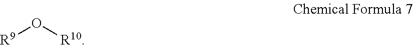
and
wherein, in Chemical Formula 7,
R⁹ and R¹⁰ are each independently a substituted or unsubstituted C3 to C20 alkyl group.

12. The resist topcoat composition of claim 11, wherein the ether-based solvent is selected from:
diisopropyl ether, dipropyl ether, diisoamyl ether, diamyl ether, dibutyl ether, diisobutyl ether, di-sec-butyl ether, dihexyl ether, bis(2-ethylhexyl) ether, didecyl ether, diundecyl ether, didodecyl ether, ditetradecyl ether, hexadecyl ether, butyl methyl ether, butyl ethyl ether, butyl propyl ether, tert-butyl methyl ether, tert-butyl ethyl ether, tert-butylpropyl ether, di-tert-butyl ether, cyclopentylmethyl ether, cyclohexylmethyl ether, cyclopentylethyl ether, cyclohexylethyl ether, cyclopentylpropyl ether, cyclopentyl-2-propyl ether, cyclohexylpropyl ether, cyclohexyl-2-propyl ether, cyclopentylbutyl ether, cyclopentyl-tert-butyl ether, cyclohexylbutyl ether, cyclohexyl-tert-butyl ether, 2-octanone, 4-heptanone, and combinations thereof.

13. A method of forming patterns, the method comprising:
forming a photoresist pattern on a substrate,
coating the resist topcoat composition of claim 1 on the photoresist pattern,
drying and heating the substrate on which the resist topcoat composition is coated to form a topcoat, and
spraying a rinse solution on the substrate coated with the topcoat to remove the topcoat.

14. The method of claim 13, wherein the heating of the substrate coated with the resist topcoat composition is performed at a temperature of about 100° C. to about 500° C.
